(19) United States
(12) Patent Application Publication
(10) Pub. No.: US 2010/0042154 A1
(43) Pub. Date: Feb. 18, 2010

(54) FLEXIBLE STABILIZATION DEVICE INCLUDING A ROD AND TOOL FOR MANUFACTURING THE ROD

(76) Inventors: Lutz Biedermann, VS-Villingen (DE); Wilfried Matthis, Weisweil (DE); Martin Pabst, Donaueschingen (DE); Berthold Dannecker, St. Georgen (DE)

Correspondence Address: CHRISTIE, PARKER & HALE, LLP, PO BOX 7068, PASADENA, CA, 91109-7068, US

(21) Appl. No.: 12/539,239

(22) Filed: Aug. 11, 2009

Related U.S. Application Data
(60) Provisional application No. 61/088,276, filed on Aug. 12, 2008.

(30) Foreign Application Priority Data
Aug. 12, 2008 (EP) 08 014 378.7

(51) Int. Cl. A61B 17/70; B26F 1/14; B23P 13/04

(52) U.S. Cl. 606/254; 83/684; 29/558

(57) ABSTRACT

A flexible stabilization device for connecting at least two bone anchoring devices which are attached to vertebrae of the spinal column includes a flexible rod made of an elastomer material. The rod has a surface in which at least one recess is provided which is generated by separation of a portion of said elastomer material.

### CROSS-REFERENCE TO RELATED APPLICATION(S)

The present application claims the benefit of U.S. Provisional Patent Application Ser. No. 61/088,276, filed Aug. 12, 2008, the contents of which are hereby incorporated by reference in their entirety, and claims priority from European Patent Application EP 08 014 378.7, filed Aug. 12, 2008, the contents of which are hereby incorporated by reference in their entirety.

### BACKGROUND

The present application relates to a flexible stabilization device for connecting at least two bone anchoring devices which are attached to vertebrae of the spinal column.

Such flexible stabilization devices may generally include a rod which is provided with a considerable degree of stiffness in order to stabilize the spinal column. The bone anchoring devices may include receiving parts, which are provided with recesses to receive the rod, and fixation screws to accomplish a tight connection between the receiving parts and the rod. The receiving parts are further connected with, or integrally formed with bone screws, which may be screwed in to the adjacent vertebrae, e.g., pedicles. Thereby, multiple anchoring devices may be connected using the rod as described above.

In recent years there have been many efforts to provide a connection rod with flexible behaviour. The flexibility allows the spinal column to be moved in a controlled manner.

As an example, WO 1996/016608 A1 shows a vertebral instrumentation rod which is made of a basically rigid material, i.e., a metal or metal alloy. Hence, the rod is rigid in a first part where a cross section is generally cylindrical. However, in a second part, the rod is substantially flat to allow for flexion in a sagittal plane while impeding flexion in the frontal plane. In a transition zone, the degree flatness smoothly increases from the first towards the second part.

US 2005/085815 A1, by Applicant, shows a rod-shaped element wherein a flexible section is integrally formed with two adjacent rigid sections. The rod-shaped element may include a metal material, and the flexible section may be provided by a coil spring allowing for flexion. In one specific embodiment, a cross section of the flexible section, i.e., of the coil spring, is flattened in one direction in achieve desired flexion properties.

US 2007/049937 A1, by Applicant, shows in one embodiment a rod-shaped implant element wherein an flexible section is connected between stiff portions. The flexible section is made from, e.g., polyurethane or polysiloxane, whereas the stiff portions are made from, e.g., titanium. The connection is provided by threads. Further, the flexible section may have a reduced or increased thickness depending on a desired amount of compression or extension capabilities.

US 2007/270821 A1 shows a vertebral stabilizer, which includes a connector formed as a single piece construction. The connector has an annular section of a reduced diameter, as compared with interposed sections, in order to enable stretching of the same. The connector may be formed from flexible fiber material.

US 2003/191470 A1 shows a dynamic fixation system wherein a rod may be shaped and thinned to function as a spring or pivot. The rod may be connected with pedicle screws via connectors while elastomer materials may be used for the rod, although not being preferred. A stepped flattened cross sectional profile may be obtained depending on the desired flexion or torsion characteristics.

Based on the foregoing, there is a need to improve known techniques of stabilizing human vertebrae and to provide a flexible stabilization device that covers desired flexion and torsion characteristics while reducing the efforts and costs of manufacturing the same.

### SUMMARY

According to one aspect of the invention, a flexible stabilization device includes a flexible rod made of an elastomer material, and a surface of the rod having at least a first recess that is formed by separating material from the rod.

The recess may influence the local bending and/or torsional properties of the rod. In one aspect, the separation may yield a directed removal of material from the rod along a first direction. Hence, the recess formed accordingly may affect the flexural rigidity of the rod in one particular (second) direction, while the original flexural rigidity of the rod may be maintained in another direction, e.g., perpendicular to the first direction.

Therefore, the recess applied to the rod may serve to achieve a reduced flexural rigidity of the rod along an orientation direction according to the specific needs of the concerned spinal column. For example, the overall flexural rigidity of the rod may be reduced at a specific position along the spinal column in order to allow for an improved flexion in, e.g., the frontal or the sagittal plane.

In one specific embodiment, the separation process refers to punching out material from the rod using a punching or cutting tool. This further allows implementing multiple punched recesses to be formed along the rod surface considering local flexural strength and the orientation of the local flexural strength with respect to the rod, or spinal column to obtain a desired property of the rod. According to specific embodiments of this aspect, the depth, length, mutual orientation or the number density of the multiple recesses can be varied along the rod to treat different parts of the spinal column in the most appropriate manner regarding flexion and/or torsion.

A tool according to a related aspect includes a socket die with a first bore for receiving the rod connector, and at least one second bore for receiving a first punching press, and further includes the punching press, which fits within the at least one second bore being moveable with respect thereto. Thereby, the first and the at least one second bore intersect each other to enable punching out material from the flexible rod to form the recess due to the movable punching press.

By using a rod having punched recesses and employing a corresponding punching tool as described above, the shaping of the rod can be accomplished by the surgeon or another person prior to the actual surgery, since no complex injection molding method is required. The tool can also be used easily in the operating room environment.

### BRIEF DESCRIPTION OF THE DRAWINGS

FIG. 1 shows a schematic drawing of the spinal column, to which is added a stabilization device including a set of bone screws and one of three types of rods;

FIG. 2 shows a rod in including recesses according to an embodiment in a perspective view;

FIG. 3 is the same as FIG. 2, but in a top view, the rod being rotated by 90 degrees between the states shown;

FIG. 4 shows a perspective view of a tool for separating material from an elastomer rod according to a first embodiment in a state before separation;

FIG. 5 is the same as in FIG. 4, but in a state when the tool is operated to separate the material;

FIG. 6 is the same as in FIG. 4, but in a cross-sectional representation;

FIG. 7 is the same as in FIG. 5, but in a cross-sectional representation;

FIG. 8 shows in a perspective view a second embodiment of a tool for separating material from an elastomer rod, wherein the state shown refers a first step of a separation method;

FIG. 9 is the same as FIG. 8, but with reference to a second step;

FIG. 10 is the same as FIG. 8, but with reference to a third step;

FIG. 11 is the same as FIG. 8, but with reference to a fourth step;

FIG. 12 shows further embodiments of recesses which may be implemented with a flexible rod by a separation method.

FIGS. 13a and 13b show a perspective and top view, respectively, of a rod according to embodiments of the disclosure.

FIGS. 14a and 14b show two embodiments of a cross-section of a rod taken at section AB of FIG. 13b.

### DETAILED DESCRIPTION

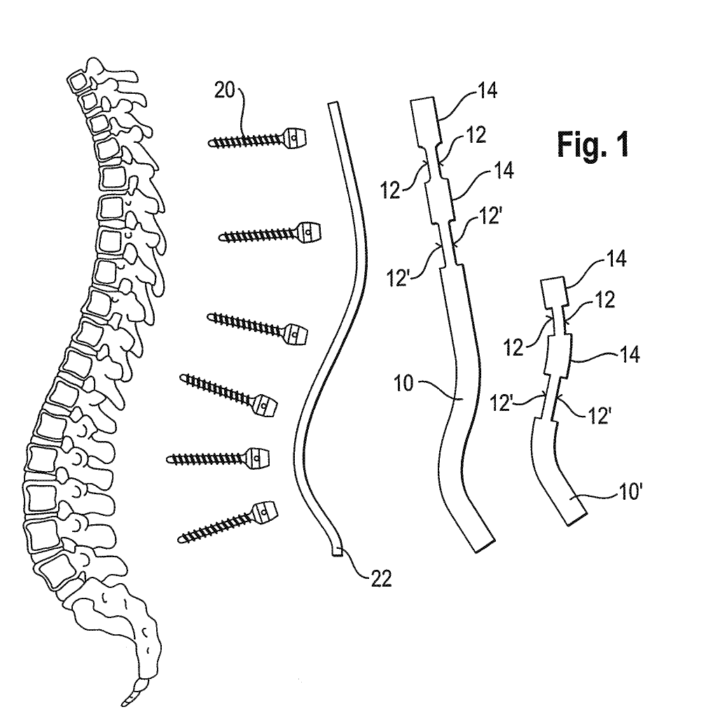

FIG. 1 shows in a schematic drawing an example of the spinal column, to which a dynamic stabilization device according to one aspect of the disclosure may be attached.

In a dynamic stabilization device, a flexible rod 22 may be employed to provide a fusion to vertebrae of the spinal column when the rod is clamped by respective bone anchoring devices 20. Thereby, the bone anchoring devices 20 are screwed into specific vertebrae at appropriate height positions selected by the surgeon. The bone anchoring devices may be one of the monoaxial type bone anchoring devices (i.e., the bone thread part and receiving part are rigidly fixed to each other) or the polyaxial type bone anchoring devices (i.e., the bone thread part being pivotable with respect to an axis of receiving part prior to locking), but the present embodiments shall not be limited to the specific functions of the bone anchoring device.

The rod 22 which is schematically indicated in the center portion of FIG. 1 includes a constant diameter or thickness throughout its length and is made of an elastomer material. The thickness is chosen such as to provide a reasonable degree of stiffness or rigidity over its entire length. When this rod is clamped by corresponding receiving parts of the bone anchoring devices, a substantially rigid fusion between the vertebrae involved is achieved.

However, in various instances, it may be desirable to increase the flexibility of the rod. In one instance, the load which acts on vertebrae in a direct neighbourhood of fused parts of the spine may be too high. In order to relieve the load, the end part of the fusion can be provided with an enhanced degree of flexibility to enable a slight bending movement of the respective vertebrae.

Hence, in the present embodiment, a rod 10 made of an elastomer material is manufactured from the rod 22, wherein the rod 10 now includes recesses 12 formed on opposite sides of the rod. The recesses are formed by removal of material from the rod 22. Due to this removal, the rod is thinned, as a result of which the bending flexibility increases locally. The outermost vertebrae are thus allowed to perform a slight movement depending on the load.

The opposing recesses 12 are formed between two respective clamping sections 14 of the rod 10, which are defined such as to be received by the receiving parts of respective bone anchoring devices 20 and thus include an appropriate length section of the rod 10.

In order to allow further portions of the spine to undergo a slight bending movement, an additional pair of recesses 12′ is formed in the rod surface. The opposing recesses 12′ may leave a same thickness of the rod as the recesses 12, while the length may be slightly extended, as an example.

In FIG. 1 there is also shown a rod 10′ as an alternative embodiment which has the same features as the rod 10, but is provided with a comparatively shorter length.

The rod according to the embodiments includes a preferably bio-compatible elastomer material. Examples of such materials which may be embodied herein are polyurethane, polysiloxane, Poly(styrene-block-isobutylene-block-styrene) (SIBS), or polycarbonate urethane (PCU).

It may be noted that the term “rod” as used herein basically denotes a rod-shaped element, which may be provided as a single piece rod as well as a multi-part composite element, that is put together to yield a fusion. In the latter case, those parts may for example be provided with corresponding threads to connect the corresponding pieces. Further, one of the parts may include the elastomer material while another part of the rod may include, e.g., a metal.

A case in which for example a single piece rod is formed from injection molding of two or more different elastomer material components shall also be encompassed by the invention, wherein the recesses are applied afterwards.

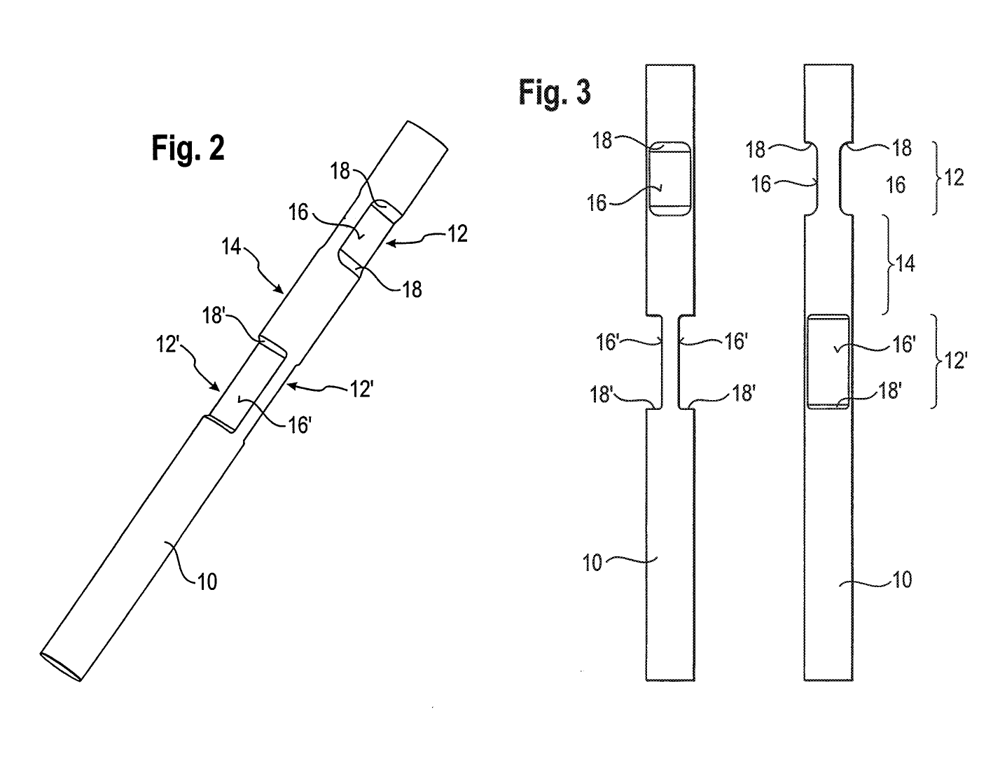

FIGS. 2 and 3 show another embodiment of a rod 10 which also includes an elastomer material similar to that shown in FIG. 1. However, unlike in the previous embodiment the recesses 12 and 12′ (or pair of recesses) are oriented in different directions with respect to each other by about 90 degrees.

The recesses 12, 12′ have a well-defined shape of limited length. As becomes evident from FIG. 3, the recesses 12 include a flat portion 16 and two rounded end portion 18. The further recesses 12′ also have a flat portion 16′, while the end portions 18′ are steeper and rise up sharply. In this embodiment, the flat portions 16′ have a larger depth with respect to the surface than flat portions 16.

It may be noted that a rod 10 as described herein may also be formed with multiple recesses 12 or 12′, all of which have the same shape—possibly with orientations with respect to the longitudinal axis of the rod, which differ from each other.

It may be noted that if a recess is formed by later removal of material, this later removal of material may in some instances be recognizable from the product. For example, when the recess is formed by punching, due to a slight deformation or flow of stressed elastomer material during punching and/or heating, plane surfaces or straight lines which are formed thereby also may become slightly concave or convex, respectively. Further, score marks or miniature rims may form across the cut surface along the punching direction.

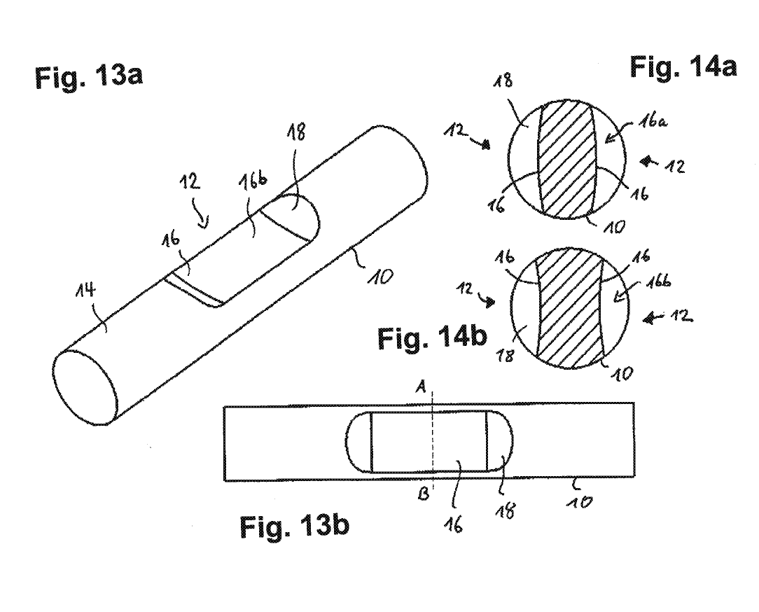

Examples of the slightly concave or slightly convex portions of the recess are illustrated in FIGS. 13a-b and 14a-b. Two examples of the cross-section of the rod 10 taken at section AB of FIG. 13b are shown in FIGS. 14a and 14b. The shape of the recesses 12 on opposing sides of the rod as shown in FIGS. 14a and 14b may result from separating material from the rod with a punching operation. In the example shown in FIG. 14a, the surfaces of flat portions 16 have a slightly convex shape 16a. In the example shown in FIGS. 13a and 14b, the surfaces have a slightly concave shape 16b. The formation of the curved surfaces and degree of concavity or convexity depends on the properties of the elastomeric material such as elasticity, the sharpness of the cutting tool of a punching press, and/or the geometry of the back support holding the rod within the punching press. For example, the noted curvatures may be less or hardly visible if sharper blades, a back support shaped to complement the shape of the rod, and/or a comparatively more rigid elastomeric material are used. Thus, forming the recesses by a punching operation may result in the recesses 12 having slight curvatures rather than having geometrically flat surfaces depending on the material properties of the rod and the characteristics of the tool that is used to form the recesses in the rod.

Despite the above mentioned possible presence of concave surfaces, score marks or miniature rims, it has been found that a punching operation may still fulfil the requirements with respect to surface roughness and punching accuracy quite satisfactorily, if a tool for punching a rod according to another aspect of the invention is used.

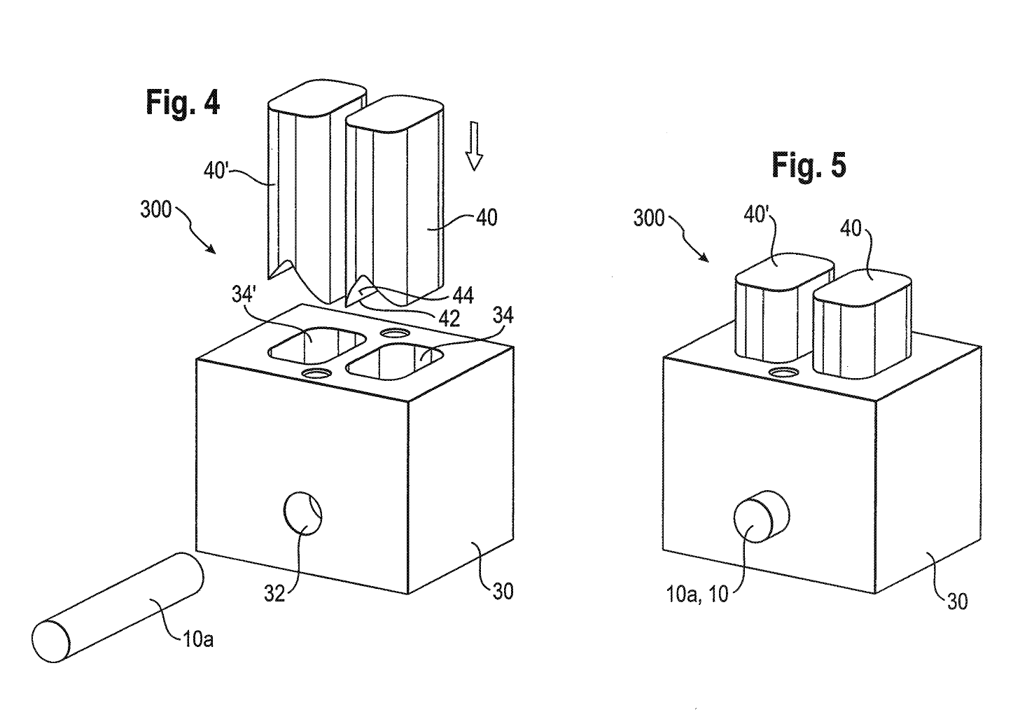

FIGS. 4-7 show a first embodiment of a tool 300 for removing elastomer material from a rod 10a to manufacture one of the rods 10 shown in FIGS. 1-3. The tool shown represents a punching tool. The tool includes a socket die 30, which is provided with a bore 32 for insertion of the yet un-punched rod 10a, and with bores 34, 34′ which receive each one of two punching presses 40, 40′. In this embodiment, the bore 32 extends through the socket die 30 in a horizontal direction while bore 34, 34′ are oriented in a vertical direction. The rod 10a can be freely adjusted—i.e., pushed or rotated—in its position within bore 32 wherein the outer diameter of the rod 10a substantially corresponds to the inner diameter of the bore 32.

The punching presses 40, 40′ are also shaped and sized to fit into the bores 34, 34′. As the bores 34, 34′ are formed in parallel to each other, the orientation and guidance of the punching presses 40, 40′ are also parallel. The bores 34, 34′ intersect with the bore 32 such that the punching presses 40, 40′ may cut material from rod 10a inserted in the bore 32. For this purpose, both presses are provided with cutting edges 42, 42′ and cutting faces 44, 44′ whose rake angle relative to the cutting direction serves to separate the removed elastomer material from the rod. The removed material may be discharged into a container not shown in the figures.

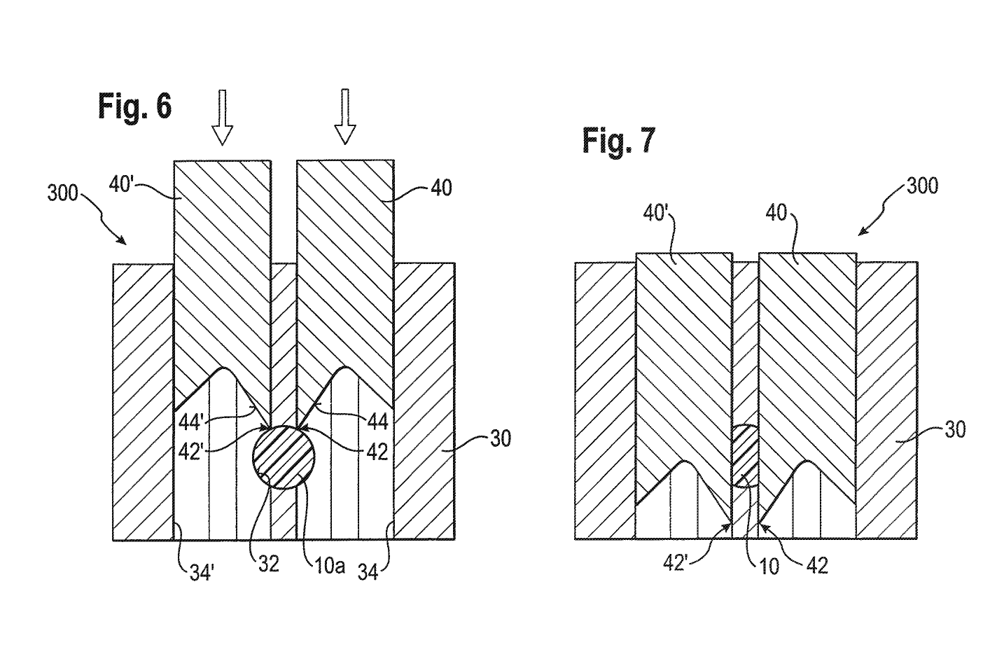

Upon exerting a load on the flexible rod, an inevitable deformation or flexible flow of material will occur in the rod. As depicted in the cross sections of FIGS. 6 and 7, a simultaneous downward movement of the punching presses helps to develop a symmetric deformation of the flexible material of the rod during the punching operation. Since bores 34, 34′ intersect with the bore 32 off-center from its longitudinal axis on opposite sides thereof, recesses such as shown in FIGS. 1-3 may be formed in the rod surface. Both punching presses may therefore be mechanically coupled with each other and further with an operating device. Such operating device may for example be a simple toggle joint. However, any other operating device capable of pressing down the punching presses with suitable force may be employed as well. Thus, according to the disclosure, cutting tools including one or more punching presses may be used.

The separating tool may be placed or installed in the vicinity of surgeon's operating site, i.e., in a hospital. Accordingly, the surgeon or an attending person may in situ decide where and to what extent recesses shall be applied to the rod. Hence, costs and efforts can be reduced which are necessary to provide a flexible stabilization device suited for the specific needs of a patient.

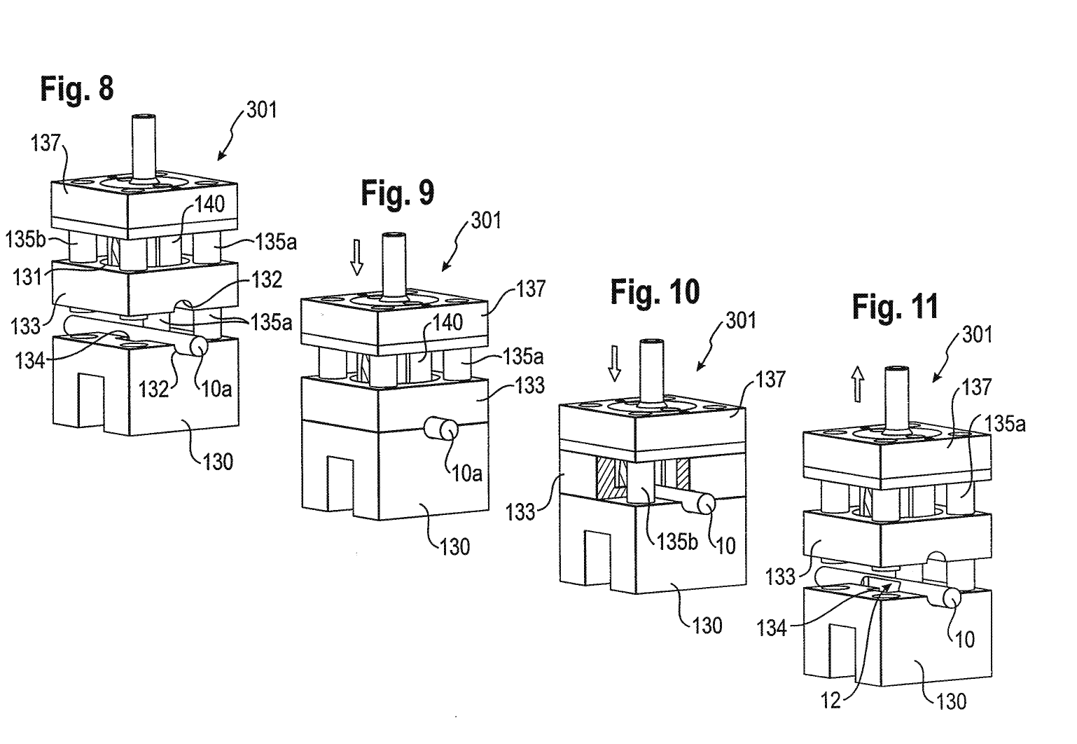

FIGS. 8-11 show another embodiment of a tool 301 for removing material from a rod 10a. This embodiment differs from the previous embodiment particularly in that the tool includes a structure which includes three plates 130, 133, 137. Other parts not described in detail herein and the shape, orientation and function of the presses can be the same as in the embodiment of FIGS. 4-7.

The bottom die plate 130 includes one half of a bore 132 designed to receive the yet un-punched rod 10a. A presser plate 133 is supported by a first spring device (e.g., a coil spring not shown) in a distance above the bottom die plate 130 and includes the other half part of the bore 132 in a bottom face thereof. When the presser plate 133 moves down until the upper face of the bottom die plate 130 is contacted, the rod 10a is held fixed inside the bore 132.

Two guide rods 135a extend upwards from the bottom die plate 130. The presser plate 133 has corresponding bores which receive the guide rods such that the presser plate 133 is held to be movable up and down along the guide rods 135a. The die plate 130 and the presser plate 133 correspond to the socket die of the previous embodiment.

A cutting plate 137 supported in a distance above the presser plate is also guided by the guide rods 135a. The distance between plates 133, 137 is maintained by a second spring device whose spring force is larger than that of the first spring device. Two further guide rods 135b extend downward from the cutting plate 137 in order to be received by corresponding bores of presser plate 133. The presser plate 133 is also movable with respect to the guide rods 135b.

Similar to the previous embodiment, the cutting plate 137 further has two cutting presses extending downward through an opening 131 of the presser plate 133 towards corresponding bores 134 formed in the bottom die plate 130. Bores 132 and 134 intersect each other as in the embodiment shown in FIGS. 6-7.

The tools shown in FIGS. 4-11 may be fabricated from stainless steel or other suitable materials.

A method of manufacturing a rod 10 as shown in FIGS. 1-3 using the tool as described above is now explained with reference to FIGS. 8-11:

First, as shown in FIG. 8, the tool is in an uncompressed state, wherein the rod 10a can be inserted into the opened bore 132 on a top face of the bottom die plate 130.

Next, as shown in FIG. 9, the cutting plate 137 is moved down using, e.g. a toggle joint, etc. Since the second spring device requires a larger force to be compressed than the first spring device, the presser plate moves down along the guide rods 135a until the presser plate contacts the bottom die plate 130 such that the rod 10a is fixed inside the bore 132.

Next, as shown in FIG. 10, the cutting plate is further moved down also against the force of the second spring device. Eventually, the cutting presses 140 along their travel through the bores 134 contact the rod 10a (which is now held fixed by the presser plate) and remove an amount of elastomer material from the rod.

Next, as shown in FIG. 11, the load manually exerted on the cutting plate 137 is removed such that the tool 301 returns to the uncompressed state, leaving behind a rod 10 which includes recesses 12. In order to apply further recesses 12 or 12′, the rod can then be newly positioned, i.e., shifted and/or rotated.

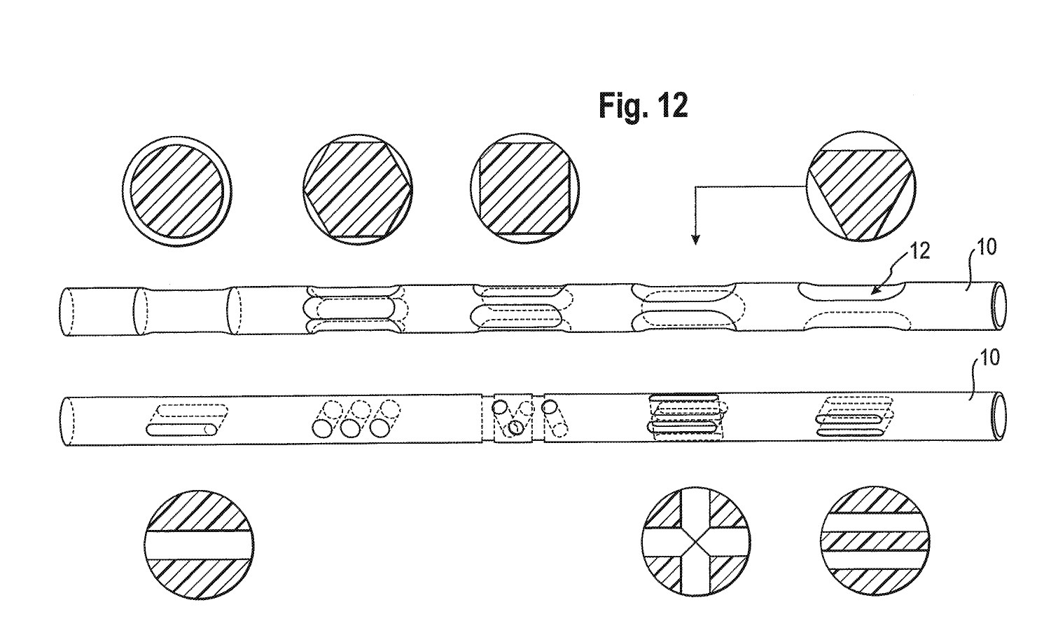

The shapes and sizes of recesses formed in the rod by removing material are not limited to those examples shown in the embodiments above. As shown in FIG. 12, which indicates various kinds of recesses in perspective view along with the associated cross sections of the rod, the recesses may be formed by removing material (a) from the periphery of the rod (upper section of FIG. 12) to yield grooves, or (b) from inner through holes extending through a rod (bottom section of FIG. 12).

Further, the separation of elastomer material from the rod as proposed herein is not limited to a cutting or punching operation. As shown in the upper left section of FIG. 12, a recess may also be formed by a turning operation in order to yield a rotationally symmetric recess. Still further, the directed removal of material as it is achieved in a punching operation may also be accomplished by milling, drilling, water cutting and/or laser cutting. Further, heat may be applied to locally melt and then remove material from the rod.

The rod can have any cross-section in sections other than those having the recesses, e.g., cylindrical, hexagonal, square, etc.

According to the invention, recesses are applied to a rod of a flexible stabilization device by removal of material in selected areas. Hence, clamping sections can be maintained between the recesses which may serve to be clamped by bone anchoring devices. The desired bending properties of the rod are thus concentrated in these selective areas outside the clamping sections. As a result, conventional abrasion of elastomer material due to the grinding of a bending rod surface inside a rigid receiving part can be considerably reduced. Consequently, the endurance of the rod can be prolonged.

While a particular form of the disclosure has been illustrated and described, it will be apparent that various modifications can be made without departing from the spirit and scope of the disclosure. Accordingly, it is not intended that the disclosure be limited, except as by the appended claims.

What is claimed is:

1. A flexible stabilization device for connecting at least two bone anchoring devices which are attachable to vertebrae of the spinal column, comprising:
a flexible rod made of an elastomer material;
the rod having a surface in which at least one first recess is provided which is generated by punching out a portion of said elastomer material from the rod-shaped element.

2. The flexible stabilization device according to claim 1, wherein the first recess is provided as a groove comprising a flat portion at a bottom surface thereof.

3. The flexible stabilization device according to claim 2, wherein:
a second recess is formed in the surface; and
the second recess is provided as a groove comprising a flat portion at a bottom surface thereof.

4. The flexible stabilization device according to claim 3, wherein the first and second grooves are provided on opposite sides within the same section of the rod.

5. The flexible stabilization device according to claim 3, wherein the first and second grooves deviate from to each other with regard to at least one of:
a depth of the flat portion with respect to the surface of the rod in a portion other than the recesses;
a length of the flat portion along a longitudinal axis of the rod; and
an orientation of a surface normal of the flat portion with respect to the longitudinal axis.

6. The flexible stabilization device according to claim 1, wherein the recess is provided as a through hole extending through the rod.

7. The flexible stabilization device according to claim 1, wherein:
a second recess is formed in the surface; and
the second recess is provided as a through hole extending through the rod.

8. The flexible stabilization device according to claim 7, wherein the first and second through holes deviate from to each other with regard to at least one of:
a diameter of the through hole; and
an orientation of a longitudinal axis of the bore with respect to a longitudinal axis of the rod.

9. The flexible stabilization device according to claim 1, wherein the rod comprises:
a clamping section provided for each of the bone anchoring devices, respectively, wherein the rod has a same constant cross section in each of the clamping sections; and
at least two intermediate section arranged between the two of the clamping sections, a first of the intermediate sections comprising the at least one first recess, and a second of the intermediate sections comprising a second recess having at least one differing property from the first recess.

10. The flexible stabilization device according to claim 1, wherein the rod is made from polysiloxane, polyurethane, Poly(styrene-block-isobutylene-block-styrene) (SIBS) or polycarbonate urethane (PCU).

11. A tool for separating material from a rod-shaped element comprising an elastomer material for use in a flexible spinal stabilization device, the tool comprising:
a socket die having:
a first bore with a first bore axis, the first bore configured to receive the rod-shaped element along the first bore axis;
a second bore with a second bore axis offset from the first bore axis, the second bore intersecting the first bore;
a third bore with a third bore axis offset from the first bore axis and offset from the second bore axis, the third bore intersecting the first bore;
a first punching press having a cutting portion, the first punching press being moveable in the second bore between a first position wherein the cutting portion is outside the first bore and a second position wherein the cutting portion is in the first bore; and
a second punching press having a cutting portion, the second punching press being moveable in the third bore between a first position wherein the cutting portion is outside the first bore and a second position wherein the cutting portion is in the first bore.

12. The tool according to claim 11, wherein the second and the third bore are arranged in parallel and partially intersect with the first bore at positions on opposing sides of the first bore axis.

13. The tool according to claim 11, wherein the first and second punching presses are connected with each other such as to provide a common punching movement.

14. The tool according to claim 11, further comprising a toggle joint mechanism to operate the movement of the first and second inner punching presses.

15. A method of manufacturing a rod-shaped element comprising an elastomer material for use in a flexible spinal stabilization device, the method, comprising:
inserting the flexible rod-shaped element in a tool; and
separating a portion of the elastomer material from the flexible rod-shaped element by punching out the portion with a punching press of the tool to form at least one recess in a surface of the flexible rod-shaped element.

16. The method according to claim 15, further comprising separating another portion of the elastomer material from the flexible rod-shaped element by punching out the another portion with another punching press of the tool to form another recess in the surface of the flexible rod-shaped element.

17. The method according to claim 16, wherein the punching presses are moved together to simultaneously form the recesses in the rod-shaped element.

18. The method according to claim 16, wherein the first recess and the second recess are formed as grooves, each groove comprising a flat portion at a bottom surface of the groove.

19. The method according to claim 18, wherein the first and second grooves deviate from to each other with regard to at least one of:
a depth of the flat portion with respect to the surface of the flexible rod-shaped element in a portion other than the recesses;
a length of the flat portion along a longitudinal axis of the flexible rod-shaped element; and
an orientation of a surface normal of the flat portion with respect to the longitudinal axis.

20. The method according to claim 16, wherein the recesses are formed on opposite sides of the flexible rod-shaped element.

21. The method according to claim 16, wherein the another recess is formed as a through hole extending through the flexible rod-shaped element.

22. The method according to claim 15, wherein the recess is formed as a through hole extending through the flexible rod-shaped element.

23. The method according to claim 16, wherein the recesses are formed as a through holes extending through the flexible rod-shaped element, and wherein the first and second through holes deviate from to each other with regard to at least one of:
a diameter of the through hole; and
an orientation of a longitudinal axis of the bore with respect to a longitudinal axis of the flexible rod-shaped element.

24. The method according according to claim 15, wherein the flexible rod-shaped element is made from polysiloxane, polyurethane, Poly(styrene-block-isobutylene-block-styrene) (SIBS) or polycarbonate urethane (PCU).
